(19) United States
(12) Patent Application Publication
(10) Pub. No.: US 2017/0127193 A1
(43) Pub. Date: May 4, 2017

(54) HEARING AID SYSTEM AND METHOD CONTAINING A SENSOR FOR CAPTURING BIOLOGICAL DATA

(71) Applicant: SIVANTOS PTE. LTD., SINGAPORE (SG)

(72) Inventor: KUNIBERT HUSUNG, ERLANGEN (DE)

(21) Appl. No.: 15/338,668

(22) Filed: Oct. 31, 2016

(30) Foreign Application Priority Data
Oct. 29, 2015 (DE) 102015221187.8

(51) Int. Cl. H04R 25/00; A61B 5/01; A61B 5/024; A61B 5/1455; F21V 23/00; A61B 5/00

(52) U.S. Cl. CPC H04R 25/505; H04R 25/604; F21V 23/003; A61B 5/6815; A61B 5/02438; A61B 5/14552; A61B 5/443; A61B 5/01; A61B 5/02433; A61B 5/0022; H04R 25/356; F21Y 2115/10

(57) ABSTRACT

A hearing aid system contains a hearing aid having with an acoustoelectric input transducer for recording a sound signal and converting the latter into an electrical input signal, a signal processing unit, connected to the input transducer, for processing the input signal, and an electro acoustic output transducer, connected to the signal processing unit, for converting the processed input signal into an acoustic output signal. A sensor is provided for capturing a measurement value of a biological measured variable and for outputting electrical information signal carrying information about the measurement value. The signal processing unit used to process the input signal is also signal-connected to the sensor and additionally configured and embodied to process the information signal. Furthermore, a method for establishing biological data of a hearing aid wearer by a corresponding hearing aid system is possible.

### CROSS-REFERENCE TO RELATED APPLICATION

This application claims the priority, under 35 U.S.C. §119, of German application DE 10 2015 221 187.8, filed Oct. 29, 2015; the prior application is herewith incorporated by reference in its entirety.

### BACKGROUND OF THE INVENTION

### Field of the Invention

A hearing aid serves to supply a hearing impaired person with acoustic signals from the surroundings, which have been appropriately processed for compensating the respective hearing impairment and, in particular, amplified. To this end, a hearing aid usually contains one or more acoustoelectric input transducers, for example in the form of a microphone, a signal processing unit with an amplifier, and an electro acoustic output transducer. The output transducer is generally realized as a miniaturized loudspeaker and is also referred to as receiver. It produces acoustic output signals which are forwarded to the ear of a patient and produce the desired audio perception for the patient.

There are various embodiments of hearing aids. In the case of so-called in-the-ear (ITE) hearing aids, a housing which contains all functional components, including the microphone and the receiver, is worn at least partly in the auditory canal. Completely-in-canal (CIC) hearing aids are similar to ITE hearing aids, but are worn completely in the auditory canal. In the case of behind-the-ear (BTE) hearing aids, a housing with components such as a battery and the signal processing unit is worn behind the ear. A flexible sound tube, also referred to as tube, guides the acoustic output signals of the receiver from the housing to the auditory canal, where a corresponding earpiece is usually provided on the sound tube for positioning the end of the sound tube in the auditory canal.

Irrespective of the configuration of the respective hearing aid, hearing aids which, in addition to the conventional function thereof as a sound amplifier for ambient noises, are also usable for different purposes are being developed ever more frequently in the meantime. Thus, modern hearing aids may, for example, be coupled without problems to telephones, televisions and/or audio systems in order to directly play-in the audio signals thereof.

Monitoring and recording vital functions of the hearing aid wearer also represents a further field of interest. In principle, such monitoring of biological data, in particular data relating to the metabolism or vital functions, by use of so-called “wearables” is known. These include e.g. armbands, watches, pieces of clothing or headphones which are used by sensors to measure blood pressure, heartbeat or the like. Then, for example during physical exercise, the vital functions may be monitored directly (in the case of watches or armbands) or (in the case of items of clothing or headphones) by of e.g. a (wirelessly) coupled smartphone (or the corresponding application).

Using the hearing device together with a system for monitoring the activities of the user, for example during physical exercise, is known from U.S. Pat. No. 8,655,004 B2. The monitoring system may be arranged in the vicinity of the head or the ear of the user and is able to measure user-specific biological data, such as the body temperature, the heartbeat or sweating. To this end, the monitoring sensor contains a sensor which may be either integrated into the corresponding hearing device or fastened thereto.

U.S. Pat. No. 5,721,783 A discloses an option for recording signals from the surroundings or biological data of the hearing aid wearer by an external apparatus such as e.g. a watch or a piece of jewelry, amplifying or improving said signals or biological data and subsequently transferring these wirelessly to a hearing aid or an earpiece of a hearing aid.

What is common to both systems described above is that the signal processing of the biological data recorded by the sensor is carried out by separate signal processing units provided specifically for this purpose, which are appropriately coupled to the respective hearing device. This is complicated and expensive.

### SUMMARY OF THE INVENTION

A first object underlying the invention is that of specifying a hearing aid system which allows a technically simplified connection of the sensor for capturing biological data to a hearing aid.

Specifying a method by which a correspondingly simplified connection of the sensor for capturing biological data to a hearing aid is possible is a second object underlying the invention.

According to the invention, the first object of the invention is achieved by a hearing aid system containing a hearing aid with an acoustoelectric input transducer for recording a sound signal and converting the latter into an electrical input signal, with a signal processing unit, connected to the input transducer, for processing the input signal, and with an electro acoustic output transducer, connected to the signal processing unit, for converting the processed input signal into an acoustic output signal, and containing a sensor for capturing a measurement value of at least one biological measured variable and for outputting an electrical information signal carrying information about the measurement value. Here, the signal processing unit used to process the input signal is also signal-connected to the sensor and additionally configured and embodied to process the information signal.

In a first step, the invention is based on the consideration that the previously required external devices for signal processing, monitoring and transfer of vital functions, medical data or, in general, biological data of a hearing aid wearer reduce the user comfort for the hearing aid wearer since he must carry further separate components on him for coupling to the hearing aid. Technically, a multiplicity of additional electronic components is required, making the coupling of the corresponding sensor unnecessarily more expensive.

In a second step, the invention considers the fact that a signal processing unit is installed in each hearing device. The signal processing unit is used for processing and amplifying the electrical input signals generated from the sound signals.

Finally, in a third step, the invention recognizes that the signal processing units used in the hearing aid in principle have the potential to be additionally used for processing further data and, in particular, for processing signals from sensors for capturing biological data. By way of example, the frequency range below or above the faculty of hearing typically remains unconsidered when processing audio signals, even though processing of such signals is possible due to the parameters of the components used in the signal processing unit of a hearing device.

Taking this into account, the invention provides for the signal processing unit of the hearing aid to also be used for processing the electrical information signals of the sensor for capturing biological data in addition to the processing of audio signals. To this end, the corresponding sensor, by which the respective biological data are captured, is signal-connected to the signal processing unit of the hearing aid such that the signal processing unit is enabled to process both the electrical input signal from the acoustoelectric input transducer and an electrical information signal from the biosensor.

In other words, an electrical information signal from the biosensor is processed by using a signal processing unit already available in the hearing device. Previously unused capacities of the signal processing unit could be effectively used in this manner. It is possible to dispense with additional electronic components and/or a separate signal processing unit for connecting a biosensor to a hearing aid.

The sensor or biosensor captures a measurement value of the biological measured variable. By way of example, the pulse or the heartbeat, the body temperature or sweating are able to be captured as measured variables. It is also possible to measure oxygen saturation, blood sugar levels, blood pressure or further medical parameters. The electrical information signal of the sensor carrying the information about the measurement value may be a voltage, a current, a resistance, a capacitance, an inductance or the like. The sensor outputs the electrical information signal carrying information about the measurement value, the electrical information signal being processed further accordingly in the signal processing unit of the hearing device. In a particularly preferred configuration, the sensor is connected to a voltage supply unit in the hearing aid.

In an advantageous variant, the sensor is arranged on the hearing aid or integrated into the hearing aid. In particular, the hearing is embodied as an ITE hearing aid in this case. Biological data of the hearing aid wearer can be captured immediately on or in the ear by way of a sensor arranged on a concha hearing aid or on a CIC hearing aid. In another preferred configuration, the biosensor is line-connected to the hearing aid. In particular, in that case, the sensor is assembled in an earpiece of the hearing aid which is able to be inserted into the ear, the hearing aid being connected by way of e.g. a sound tube to a BTE hearing aid in such a case. In the variant of the BTE hearing aid, the receiver or the electro acoustic transducer is inserted into the earpiece and coupled in a wired manner by way of a connection tube. Then, this is also referred to as a receiver-in-canal (RIC) hearing aid. These variants have the advantage of a very simple placement of the sensor. The sensor is simply arranged with the hearing aid or with the earpiece on the position on the body of the hearing aid wearer suitable for tapping the biological data. A complicated separate attachment of the sensor at another position is dispensed with.

In an advantageous configuration of the invention, the signal processing unit contains an amplifier, in particular preamplifier, and a processor unit disposed downstream of the amplifier, with the actual processing of the amplified signal being carried out in the processor unit on an analog and/or digital basis. An amplifier unit and a processor unit are conventional components of a signal processing unit typically installed in a hearing aid. In this case, the amplifier may be embodied for digital and/or analog amplification of the signal. Accordingly, an analog-to-digital converter is implemented where necessary. If the hearing aid contains a telecoil for inductive coupling of audio signals or for inductive coupling with a second hearing aid, e.g. in the case of the binaural hearing system, the hearing aid generally contains a separate preamplifier which lifts the coupled-in signals to the signal level of the input signals coupled in by way of the acoustoelectric input transducer. In a preferred variant, the information signal of the biosensor is conducted via the preamplifier of the telecoil before it is further amplified by an amplifier and processed in the processor unit. This is particularly advantageous in the case of a biosensor which supplies very weak information signals.

In principle, it is possible for the information signal and the input signal to be supplied independently of one another to the signal processing unit, in particular to the amplifier and/or the processor unit. However, it is advantageous for the information signal from the biosensor to be applied to the input signal from the acoustoelectric input transducer on the input side or for both signals to be coupled such that the signal processing unit is used for common processing, in particular for common amplification, of both signals. Preferably, at least both signals are amplified together. This is possible since the frequency components of the audio signal and of the information signal of the biosensor typically differ. Optionally, as mentioned above, the information signal is initially conducted via the preamplifier of the telecoil before it is applied to the input signal.

Expediently, a separation unit for separating the common amplified signals is interposed between the amplifier and the processor unit in the signal processing unit. The separation unit is configured and embodied to separate the signals amplified together by means of the amplifier. After separating the signals amplified together, the audio signal and the information signal from the biosensor are subsequently processed separately.

Preferably, the processor unit contains an audio processor unit for processing the input signal and/or a bio-processor unit for processing the information signal for the purposes of separate processing of the audio, or input, signal and the information signal. The audio processor unit assumes the preparation of the audio signal for the hearing aid wearer. The prepared audio signal is accordingly output to the electro acoustic output transducer for the purposes of outputting a sound signal. The electrical information signals which are established by the biosensor and optionally amplified are processed by the bio-processor unit in such a way that they are usable for a hearing aid wearer. Thus, in a preferred configuration, the information signals are e.g. prepared so as to be displayed on an external output unit, for example a smartphone or the like. By way of example, the vital functions of the respective user are displayed graphically (for example on the basis of a heart rate symbol on the display of the smartphone) after a corresponding transfer of the prepared information signals. Warnings may also be prepared, output and transmitted. Here, the connection between the bio-processor unit and the respective output unit is preferably realized in a wireless manner, in particular by way of Bluetooth or by way of WLAN. To this end, the bio-processor unit is coupled to an antenna of the hearing aid in an advantageous variant, the antenna facilitating a corresponding wireless connection to an external device.

Warning messages, sounds or else voice announcements in relation to the measurement values of the biosensor or in relation to evaluations may also be output in the output transducer of the hearing aid. To this end, it is preferable for the bio-processor unit to be connected to the audio processor unit for the purposes of an acoustic signal output in one configuration variant. The electrical information signals processed in the bio-processor unit are appropriately prepared for the audio processor unit, processed further by the latter and forwarded to the electro acoustic output transducer of the hearing aid for outputting an acoustic signal. Thus it is possible, for example on a permanent basis or when necessary, to output the heart tone to a hearing aid wearer by way of the hearing aid, to state the heart rate or other biological parameters, or to directly provide information or a warning by way of a signal tone or a corresponding announcement in the case of physical overexertion or when a normal range is left.

The use of the amplifier for common amplification of the electrical information signal from the sensor and the electrical input signal from the acoustoelectric input transducer is possible, in particular, if the audio signal and the information signal from the bio-sensor have frequency ranges that differ from one another. Here, the information signal from the sensor is expediently variable over time and substantially contains frequencies from a frequency range lying below the frequency range of the input signal. Preferably, the information signal of the sensor contains frequencies in a frequency range below 10 Hz. The input signal typically contains frequencies in a frequency range above 100 Hz.

The different frequency ranges firstly facilitate a common amplification in the signal processing unit of the hearing aid, without requiring additional measures. Secondly, a separation of the signals by frequency selection is possible after amplification has been carried out. To this end, the separation unit used for separation is preferably embodied for frequency-selective splitting of the common amplified electrical signals. By way of example, the separation unit is embodied as a crossover and divides the signal into various frequency ranges, i.e. into the information signal and the audio signal. The separated signals are then processed separately in the signal processing unit, in particular by the audio processor unit and the bio-processor unit.

The sensor or biosensor is preferably embodied as an optical sensor and, in this case, expediently contains at least one light source and a detection unit. An LED is preferably used as light source. The light emitted by the LED is detected by the detection unit, which is preferably embodied as a photodiode. The signal detected by the optical sensor is preferably a voltage or current signal. Expediently, a filter is disposed downstream of the sensor, the filter serving to pre-filter the signals for separating out disturbance, noise or background signals before said signals are supplied to the signal processing unit.

The sensor is particularly preferably configured and embodied to capture a heartbeat of a hearing aid wearer. A sensor embodied to capture the heartbeat contains e.g. an infrared-emitting diode (IR-LED), by which the signals reflected by a pulsating artery are measured. The capillaries expand and contract again with each heartbeat due to changes in the blood volume. The light of the IR-LED reflected by the skin captures these changes. By way of example, the reflected light is detected by a photodiode. Then, as described above, the information signals are amplified and processed further. In the case of the heartbeat, the information signal of the sensor is a periodic or time-varying signal.

In another configuration, the oxygen saturation in the blood of the hearing aid user is captured by an optical sensor. In particular, a sensor embodied to capture the oxygen saturation in the blood has two light-emitting diodes (LEDs) which differ in terms of the emission wavelengths thereof. In particular, a light-emitting diode (R-LED) emitting red in the visible spectral range and an infrared light-emitting diode (IR-LED) serve as light sources for the measurement. The pulsation of the arterial blood flow is also used to determine the arterial oxygen saturation, the pulsation changing the blood volume during the systole (contraction phase and hence blood outflow phase of the heart) and the diastole (relaxation phase and hence blood inflow phase) and hence acting on the light absorption. A change in the light absorption is captured by way of a change in the back-reflected light. Since it is only the change in the light absorption that is evaluated, non-pulsating absorbing substances such as tissue, bones and venous blood have no effect on the measurement. In particular, in the back reflection, the sensor measures the ratio of the red pulsating absorption and infrared pulsating absorption, which is directly related to the oxygen saturation, and the sensor moreover expresses the oxygen saturation thereby.

In a further advantageous configuration of the invention, the sensor is configured and embodied to capture the skin moisture and/or the body temperature of the hearing aid wearer. The resistance of the skin changes in the case of a change in temperature or increased sweating. This change in resistance is measured, converted into an electrical signal by an appropriate transducer and finally amplified and processed further. In this case, the information signal is static. It is preferably modulated onto a periodic carrier signal with a suitable frequency for further processing or amplification.

According to the invention, the second object of the invention is achieved by a method for establishing biological data of a hearing aid wearer by a hearing aid system, a sound signal being recorded by an acoustoelectric input transducer of a hearing aid, the sound signal being converted into an electrical input signal by means of the acoustoelectric input transducer, the electrical input signal being forwarded to a signal processing unit of the hearing aid and processed there, the processed electrical input signal being forwarded to an electro acoustic output transducer of the hearing aid and being converted into an acoustic output signal there, a measurement value of at least one biological measured variable being captured by a sensor, and the sensor outputting an electrical information signal carrying information about the measurement value. Here, additionally, the information signal of the sensor is also processed in the signal processing unit used to process the input signal.

Here, the advantages specified for the hearing aid system and the advantageous configurations thereof may analogously be transferred to the method. Further advantageous variants become apparent from the dependent claims directed to the method.

Preferably, the information signal is applied to the input signal on the input side of the signal processing unit, or of an amplifier, and both signals are amplified together in an amplifier of the signal processing unit. Optionally, the information signal is initially pre-amplified in a preamplifier of a telecoil before it is applied to the input signal. Naturally, a separate supply of the two signals into the amplifier is also possible.

The, in particular amplified, electrical input signal and the, in particular amplified, electrical information signal are expediently processed in a processor unit. Here, the processor unit preferably contains an audio processor unit and a bio-processor unit, the audio processor unit processing the input signal and the bio-processor unit processing the information signal.

In an advantageous configuration, the input and information signals, amplified together, are separated from one another by the processor unit, with the input signal being processed in an audio processor unit and the information signal being processed in a bio-processor unit. The electrical input signal processed in the processor unit is preferably forwarded to the electro acoustic output transducer. The processed electrical information signal is preferably output to an, in particular external, output unit. An alternative preferred configuration provides for the processed electrical information signal to be forwarded or output from the bio-processor unit, in particular via the audio processor unit, to the electro acoustic output transducer for the purposes of outputting acoustic information.

Other features which are considered as characteristic for the invention are set forth in the appended claims.

Although the invention is illustrated and described herein as embodied in a hearing aid system and method containing a sensor for capturing biological data, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is an illustration showing a hearing aid system containing a sensor for capturing a heart rate of a hearing aid wearer according to the invention; and

FIG. 2 is an illustration showing the hearing aid system containing a sensor for capturing oxygen saturation of blood of the hearing aid wearer.

### DETAILED DESCRIPTION OF THE INVENTION

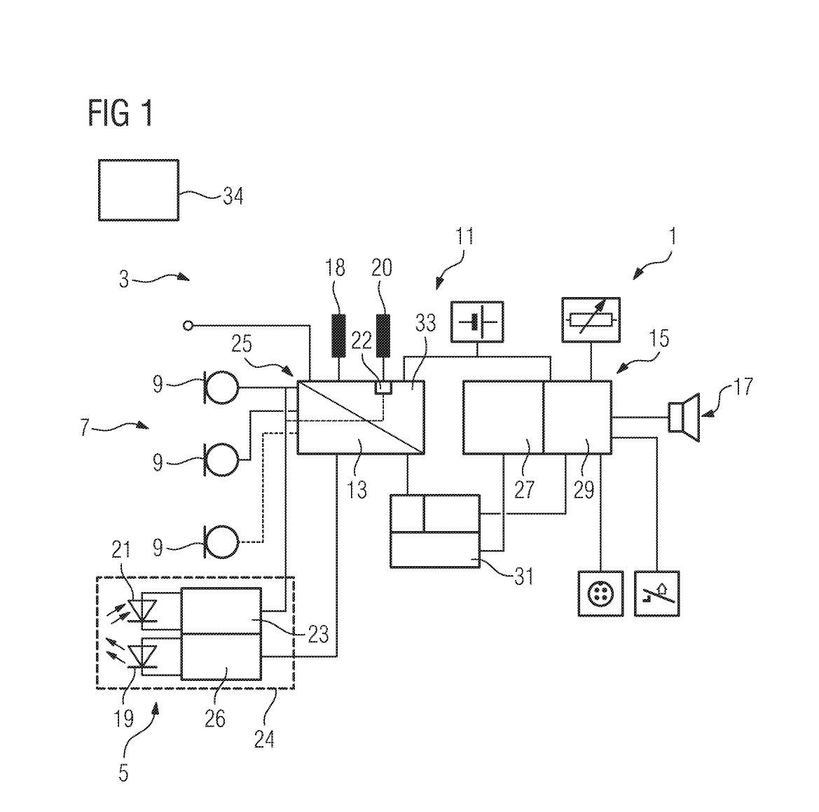

Referring now to the figures of the drawings in detail and first, particularly to FIG. 1 thereof, there is shown a hearing aid system 1 containing a hearing aid 3 and an optical sensor 5 for capturing the heart rate of a hearing aid wearer. The hearing aid 3 contains an acoustic electric input transducer 7 with three microphones 9, by which sound signals are recorded, in particular in a directionally selective manner, and converted into electrical input signals.

Furthermore, the hearing aid 3 has a signal processing unit 11, connected to the input transducer 7, with an amplifier 13 disposed upstream thereof and a processor unit 15 connected to the amplifier 13. The electrical input signals which, as audio signals, typically have frequencies in a frequency range above 100 Hz are initially amplified by the amplifier 13 for further processing. In the processor unit 15, the amplified electrical input signals are processed further, in particular amplified e.g. in a frequency-selective and/or directionally dependent manner in view of the hearing ability of the hearing aid wearer, and e.g. freed from background noise and noise. A component 33 with the amplifier unit 13 simultaneously contains an analog-to-digital converter, a transceiver for wireless coupling by way of an antenna 18 and a voltage supply unit in the present case. A preamplifier 22, in particular, is integrated in the case of a telecoil 20.

On the output side, the signal processing unit 11 is connected to an electro acoustic output transducer 17, which converts the processed electrical input signal into an acoustic output signal which is then conducted to the ear of a hearing aid wearer.

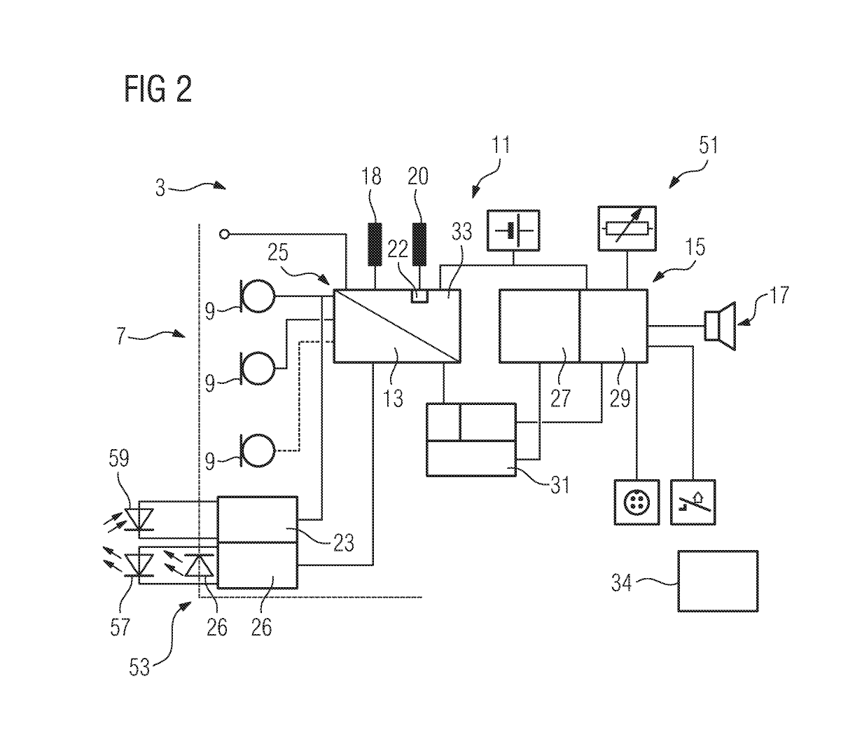

Like the acoustic electric input transducer 7 of the hearing aid, the optical sensor 5 belonging to the hearing aid system 1 is also signal connected, in particular in a wired manner, to the signal processing unit 11. In this case, the sensor 5 is also placed directly on or in the ear of the hearing aid wearer. In particular, the hearing aid 3 is embodied as an ITE hearing aid, with the sensor 5 being arranged directly on the hearing aid. This is indicated in FIG. 2. In another configuration, which is indicated in FIG. 1, the sensor 5 is arranged in an earpiece 24, with the earpiece 24 being line-connected to the hearing aid 3 and the latter being embodied as a BTE hearing aid. In a further alternative configuration, the sensor 5 is coupled to the signal processing unit 11, for example by way of a radio link. By way of example, this is possible by way of a corresponding antenna 18, which is connected to the amplifier 13. The sensor 5 is embodied with a light source 19 embodied as an IR-LED and a photodiode as detection unit 21. In the present case, the heartbeat of the hearing aid wearer is captured by the sensor 5. For the purposes of actuating the LED, the sensor 5 contains a power management unit 26, which is supplied by way of the voltage supply unit in the component 33 of the hearing aid 3.

For the purposes of capturing the heartbeat, the LED as light source 19 emits light in the infrared spectral range, which reaches into the skin of the hearing aid wearer up to a certain penetration depth and which is partly reflected. Here, the reflection varies with the heartbeat since the blood volume of the capillaries changes periodically with the heartbeat. The reflected light is detected by the photodiode 21. The photodiode 21 outputs an electrical signal which varies periodically with the heartbeat, the electrical signal being pre-filtered in a filter 23. Here, an electrical information signal with frequencies below 10 Hz, i.e. with the frequency of the heartbeat, is filtered out.

In the present case, the electrical information signal is applied to the electrical input signal on an input side 25 of the signal processing unit 11. Both signals are amplified together by the amplifier 13 of the signal processing unit 11. In another variant, the information signal is applied to the preamplifier 22 before it is amplified together with the input signal. This is indicated by dashed lines in FIG. 1. After amplification, the two signals are separated from one another in a separation unit 27, embodied as a crossover, interposed between the amplifier 13 and the processor unit 15. Here, the separation unit separates the information signal with frequencies of less than 10 Hz from the audio signal with frequencies above 100 Hz.

After the frequency-selective split of the electrical signals are amplified together in the amplifier 13, the amplified electrical input signals with frequencies in a frequency range above 100 Hz are supplied to an audio processor unit 29 of a processor unit 15. Here, the audio signals are processed and forwarded for an acoustic output to the electro acoustic output transducer 17.

The amplified electrical information signals from the sensor 5 with frequencies in a frequency range below 10 Hz are supplied to bio-processor unit 31 of the processor unit 15. Here, the information signals of the sensor 5 are processed and forwarded wirelessly by means of an antenna 18 to an external output unit 34 via a transceiver in the component 33. By way of example, the external output unit 34 is a smartphone, on which the heart rate is made visible on the display, for example on the basis of the heart rate symbol with the measured or evaluated value.

Alternatively, or additionally, acoustic information about the heart rate is output by means of the electro acoustic transducer 17 of the hearing aid 3. To this end, the bio-processor unit 31 is connected to the audio processor unit 29. The signal processed in the bio-processor unit 31 is accordingly forwarded to the audio processor unit 29 and, from there, likewise forwarded to the electro acoustic output transducer 17. The latter also converts the processed electrical information signal into acoustic output signals, which are then conducted to the ear of a hearing aid wearer. The acoustic information signal may be the heart rate as a periodic signal. However, there may also be a voice output about the heart rate. Additionally, or alternatively, a warning message may also be output if, for example, the heart rate departs from a normal or wellness range which the hearing aid wearer preset. The hearing aid wearer will then hear warning messages or voice announcements in respect of his heart rate directly by way of the hearing aid.

FIG. 2 shows a further hearing aid system 51. The hearing aid system 51 contains the hearing aid 3 in accordance with FIG. 1, and so the detailed description in relation to FIG. 1 may analogously be transferred to FIG. 2. In the present case, the employed sensor 53 is an optical sensor for capturing the oxygen saturation of the blood of the hearing aid wearer.

In contrast to the sensor 5 in accordance with FIG. 1, the present sensor 53 comprises two light sources 55, 57. A light-emitting diode 55 emitting red in the visible spectral range and an infrared light-emitting diode 57 serve as light sources for the measurement. For the purposes of determining the arterial oxygen saturation, use is made of the pulsation of the arterial blood flow which changes the blood volume during the systole and the diastole and hence acts on the light absorption. Since only the change in the light absorption is evaluated, non-pulsating absorbing substances such as tissue, bones and venous blood have no effect on the measurement.

Measured by way of the reflection is the respective difference in the absorptions during the diastole and the peak value during the systole. Here, it is postulated that the absorption increase during the systole is only caused by arterial blood. The measurement principle is based on the fact that deoxygenated hemoglobin (Hb) in the infrared range (≈940 nm−infrared light-emitting diode 57) is absorbed less than oxygenated Hb or oxygenated hemoglobin in the red range (≈660 nm−red light emitting diode 55).

The detection unit 59 of the sensor 53 embodied as a photodiode measures the reflected light from both LEDs 55, 57. The oxygen saturation is established from the ratio of the red and infrared pulsating absorption.

The further processing of the signals in the signal processing unit 11 of the hearing aid 3, and the output to an output unit and/or the speech output in the electro acoustic output transducer 17 is carried out analogously to this description in accordance with FIG. 1.

The following is a summary list of reference numerals and the corresponding structure used in the above description of the invention:
1 Hearing aid system
3 Hearing aid
5 Biometric sensor
7 Acoustoelectric input transducer
9 Microphone
11 Signal processing unit
13 Amplifier
15 Processor unit
17 Electro acoustic output transducer
18 Antenna
19 Light source
20 Telecoil
21 Detection unit
22 Preamplifier
23 Filter
24 Earpiece
25 Input side
26 Power management unit
27 Separation unit
29 Audio processor unit
31 Bio-processor unit
33 Component
34 Output unit
51 Hearing aid system
53 Biometric sensor
55 Light source
57 Light source
59 Detection unit

1. A hearing aid system, comprising:
a hearing aid containing:
an acoustoelectric input transducer for recording a sound signal and converting the sound signal into an electrical input signal;
a signal processing unit, connected to said input transducer, for processing the electrical input signal into a processed input signal; and
an electro acoustic output transducer, connected to said signal processing unit, for converting the processed input signal into an acoustic output signal; and
a sensor for capturing a measurement value of at least one biological measured variable and for outputting an electrical information signal carrying information about the measurement value, said signal processing unit used to process the electrical input signal is also signal-connected to said sensor and additionally configured and embodied to process the electrical information signal.

2. The hearing aid system according to claim 1, wherein said sensor is disposed on said hearing aid or is line-connected to said hearing aid.

3. The hearing aid system according to claim 1, wherein said signal processing unit has an amplifier and a processor unit disposed downstream of said amplifier.

4. The hearing aid system according to claim 3, wherein the electrical information signal is applied to the electrical input signal on an input side of said signal processing unit.

5. The hearing aid system according to claim 4, wherein said signal processing unit has a separation unit interposed between said amplifier and said processor unit, said separation unit configured and embodied to separate an amplified information signal and an amplified input signal.

6. The hearing aid system according to claim 3, wherein said processor unit has an audio processor unit configured and embodied to process an amplified input signal.

7. The hearing aid system according to claim 6,
further comprising an output unit; and
wherein said processor unit contains a bio-processor unit configured and embodied to process an amplified electrical information signal and output a processed amplified electrical information to said output unit.

8. The hearing aid system according to claim 7, wherein said bio-processor unit is connected to said audio processor unit for outputting the acoustic signal output.

9. The hearing aid system according to claim 1, wherein the electrical information signal of said sensor varies in time, the electrical information signal substantially containing frequencies from a frequency range below a frequency range of the electrical input signal.

10. The hearing aid system according to claim 9, wherein the electrical information signal of said sensor substantially contains the frequencies in a frequency range below 10 Hz.

11. The hearing aid system according to claim 1, wherein the electrical input signal substantially contains the frequencies in a frequency range above 100 Hz.

12. The hearing aid system according to claim 5, wherein said separation unit is embodied for frequency-selective splitting.

13. The hearing aid system according to claim 1, wherein said sensor has a light source and a detection unit.

14. The hearing aid system according to claim 1, further comprising a filter disposed downstream of said sensor.

15. The hearing aid system according to claim 1, wherein said sensor is configured and embodied to capture at least one of a heartbeat or an oxygen saturation of blood of a hearing aid wearer.

16. The hearing aid system according to claim 1, wherein said sensor is configured and embodied to capture at least one of body moisture or a body temperature of a hearing aid wearer.

17. A method for establishing biological data of a hearing aid wearer via a hearing aid system, which comprises the steps of:
recording a sound signal via an acoustoelectric input transducer of a hearing aid;
converting the sound signal into an electrical input signal by means of an acoustoelectric input transducer;
forwarding the electrical input signal to a signal processing unit of the hearing aid and the electrical input signal being processed in said signal processing unit;
forwarding a processed electrical input signal to an electro acoustic output transducer of the hearing aid and being converted into an acoustic output signal there;
capturing a measurement value of at least one biological measured variable by means of a sensor;
outputting from the sensor an electrical information signal carrying information about the measurement value; and
processing the information signal of the sensor in the signal processing unit used to process the electrical input signal.

18. The method according to claim 17, which further comprises applying the electrical information signal to the electrical input signal on an input side of the signal processing unit and both signals are amplified together in an amplifier of the signal processing unit.

19. The method according to claim 18, which further comprises processing an amplified electrical input signal and an amplified electrical information signal in a processor unit of the signal processing unit.

20. The method according to claim 19, which further comprises separating the amplified electrical input signal and the amplified electrical information signal from one another upstream of the processor unit, the input signal being processed in an audio processor unit and the electrical information signal being processed in a bio-processor unit.

21. The method according to claim 20, which further comprises forwarding a processed input signal to the electro acoustic output transducer and in that a processed information signal is output to an output unit.

22. The method according to claim 21, which further comprises forwarding the processed information signal, via the audio processor unit, to the electro acoustic output transducer to output acoustic information.
